(19) United States
(12) Patent Application Publication
(10) Pub. No.: US 2003/0104059 A1
(43) Pub. Date: Jun. 5, 2003

(54) Controlled release tablets of metformin

(76) Inventors: Manish Chawla, Rohini (IN); Rajeev S. Raghuvanshi, New Delhi (IN); Ashok Rampal, Amritsar (IN)

Correspondence Address: Ranbaxy Pharmaceuticals Inc., Suite 2100, 600 College Road East, Princeton, NJ, 08540, US

(21) Appl. No.: 10/289,070

(22) Filed: Nov. 6, 2002

(30) Foreign Application Priority Data
Nov. 6, 2001 (IN) 1134/DEL/2001

(51) Int. Cl. A61K 31/16; A61K 9/22

(52) U.S. Cl. 424/468; 514/629

(57) ABSTRACT

Controlled-release metformin and processes for their preparation, using a combination of non-ionic and anionic hydrophilic polymers, wherein the total hydrophilic polymer concentration is at least about 16% by weight of the composition.

### CROSS REFERENCE TO RELATED APPLICATION

This application claims the benefit of Indian Patent Application No. 1134/DEL/2001, filed Nov. 6, 2001

### FIELD OF THE INVENTION

The present invention relates to controlled release tablets of metformin, and processes for their preparation.

### BACKGROUND

Controlled drug delivery applications include both sustained/extended delivery and targeted delivery on a one-time or sustained basis. Controlled release formulations can be used to reduce the amount of drug necessary to cause the same therapeutic effect in patients. The convenience of fewer and more effective doses also increases patient compliance.

Metformin, is an oral anti-hyperglycemic drug used in the management of non-insulin dependent diabetes mellitus (type 2-diabetes). Metformin is a dimethyl biguanide having the formula:

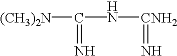

The pharmaceutically acceptable salts of the formula

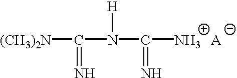

in which A is the anion of the non-toxic salt are the preferred medicaments. It is estimated that 60 percent of patients with type 2 diabetes who receive oral therapy are currently required to take doses of multiple pills several times a day in order to manage this condition. Controlled release formulation would help these patients to better control their blood sugar by making it easier to comply with their daily treatment regimen.

Metformin is a high dose drug and has poor compressibility. Therefore, the tendency of capping is particularly high during the production of tablets. It is the problem of tabletting such poorly compressible active matter, and especially those which are clinically prescribed at high dose levels that has led many workers to employ different processes to prepare controlled release formulations.

For example, Lipha (Technical Information Glucophage® August 1991, “Bundesverband der Pharmazeutischen Industrie e.v.”, Publ. Note Liste 1993, Edition Cantor, Aulendorf 1993) discloses use of framework-forming auxiliary substances such as polyvinyl acetate as retarding agents in the preparation of metformin delayed release tablets for improving the compressibility. The disadvantage of using such framework forming auxiliary substance is that they have to be processed with organic solvents. These organic solvents are not just expensive in comparison to water, but are also difficult to remove completely and therefore may lead to residual solvent in such preparations.

U.S. Pat. No. 6,117,451 provides a direct tabletting, free flowing particulate metformin hydrochloride formulation in the form of tabletting powder, capable of being directly compressed into a tablet having adequate hardness. This formulation employs specific particle size and density range excipients to improve the flow and compressibility characteristics. Use of these excipients not only adds to the cost but also makes the process cumbersome.

PCT patent application WO 99/47128 describes a method for preparing a biphasic controlled-release metformin tablet. This granulation provides the desired extended release and tackles the problem of capping but may result in segregation of granules/particles due to different particle sizes and densities during compression. Content uniformity can be difficult to achieve. Moreover, the large number of processing steps and more processing time can lead to high manufacturing costs.

U.S. Pat. No. 5,955,106 discloses a pharmaceutical composition comprising metformin and method of producing the composition wherein the composition has a residual moisture content of about 0.5 to 3% by weight, which is disclosed as critical to avoid capping of tablets.

### SUMMARY

The inventors have here addressed particular drawbacks and problems associated with the currently available technologies and provide a simple, cost effective and efficient delivery system for controlled-release metformin on a commercial scale. These objectives have been achieved by using a combination of non-ionic and anionic hydrophilic polymers, wherein the total hydrophilic polymer concentration is at least about 16% by weight of the composition.

In one aspect, controlled release tablets of metformin are disclosed which include hydrophilic polymers consisting of anionic and nonionic polymers in ratios of about 1:1 to about 1:5, and optionally other excipients, wherein at least about 16% by weight of the composition is the hydrophilic polymer. Certain embodiments have water contents of less than about 6.0%, for example, between about 3.2% and about 6.0%

In another aspect, a process is disclosed herein which comprises dry blending metformin with hydrophilic polymers consisting of anionic and nonionic polymers in a ratio 1:1 to 1:5, and optionally other excipients, granulating the blend, drying and sizing the granules, and compressing to make tablets, wherein at least about 16% by weight of the composition is the hydrophilic polymer.

Unless otherwise defined, all technical and scientific terms used herein have the same ordinary meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

In the present invention anionic and nonionic hydrophilic polymers are selected from different molecular weight sodium carboxymethylcellulose and hydroxypropyl methylcellulose, respectively.

Nonionic hydrophilic polymers can include hydroxypropyl methylcellulose with average molecular weights in the range of from about 180,000 to about 250,000, preferably about 215,000 with a degree of methoxy substitution ranging from about 19 to about 24% and hydroxypropyl molar substitution ranging from about 7 to about 12%.

Anionic hydrophilic polymers can include sodium carboxymethylcellulose, for example, with a viscosity in the range of from about 400 to about 800 cps.

Metformin is soluble in water and therefore the release of the drug from a matrix system can take place through diffusion. Therefore, controlled-release of metformin can include high viscosity polymers in the matrix system. Combination of hydroxypropyl methylcellulose and sodium carboxymethylcellulose results in rheological synergism whereby the resultant viscosity is higher than the arithmetic mean. Although the release mechanism is not limited by any particular mechanistic postulates, it is believed that a strong hydrogen bond-induced cross-linking can take place between the carboxylic group of sodium carboxymethylcellulose and the hydroxyl group of the hydroxypropyl methylcellulose.

Combination of hydroxypropyl methylcellulose with an average molecular weight in the range of from about 180,000 to about 250,000 with a methoxy degree of substitution ranging from 19 to 24% and hydroxypropyl molar substitution ranging from about 7 to about 12%, with sodium carboxymethylcellulose with a viscosity in the range of from about 400 to about 800 cps as hydrophilic polymer can be used in a mixture with metformin to give a mixture with excellent compressibility. Tablets prepared from such mixtures are hard with acceptably low friability values. Moreover, tablets produced therefrom exhibit extended release in aqueous solutions, for example, buffered solution with pH of about 6.8, of up to 12 hrs.

In addition to such polymers, the instant formulations may contain other excipients, which act in one or more capacities as, for example, diluents, binders, lubricants, glidants, colorants or flavoring agents. Diluent can not only improve the flow and compressibility characteristics of the blend, but may also aid in solving the problem of capping. However, as metformin is a high dosage drug, addition of diluent is in some cases desirable, but not necessary. If required, materials such as lactose, microcrystalline cellulose, starch, calcium hydrogen phosphate, sucrose and mannitol and the like may be used as diluent. Microcrystalline cellulose can be preferred for some particular embodiments.

Binders can be used to impart cohesiveness to the blend and also improve the flow and hardness. The polymers disclosed above could impart such properties themselves. However, excipients such as various starches, sugars, gums, low molecular weight hydroxypropyl methylcellulose and hydroxypropylcellulose may also be used as binders.

Lubricants could be used, such as those selected from talc, magnesium stearate, calcium stearate, polyethylene glycol, hydrogenated vegetable oils, stearic acid, sodium stearyl fumarate and sodium benzoate.

Glidants could also be added, such as for example, colloidal silicon dioxide (aerosil) or talc.

The Process Steps

Particular embodiments include the steps of

(a) dry blending metformin with hydrophilic polymers, such as those including hydroxypropyl methylcellulose, for example, that with average molecular weights in the range of about 180,000 to about 250,000, for example, about 215,000 with a degree of methoxy substitution ranging from about 19 to about 24% and hydroxypropyl molar substitution ranging from about 7 to about 12% and polymers such as sodium carboxymethylcellulose, for example, those with a viscosity in the range of from about 400 to about 800 cps, in the ratio of from about 1:1 to about 1:5, and optionally other excipients wherein at least about 16% by weight of the composition is the hydrophilic polymer;

(b) granulating the blend of step (a);

(c) drying and sizing the granules; and

(d) compressing the granules.

The tablets can be optionally coated using the standard coating processes. For example, it may be coated with a thin layer of a rapidly dissolving water soluble polymer or pharmaceutical excipient. In cases where a polymeric coating is required, a low molecular weight, low viscosity polymer is the preferred material. Examples of water soluble pharmaceutical excipients include lactose, sucrose, dextrose, mannitol, xylitol, and the like. In a preferred embodiment of the present invention, the water soluble excipient used as a coating is lactose. The tablets may be coated to a weight build-up of from about 1% to about 4%, preferably, from about 1% to about 2%. The coating also helps in masking any bitter taste associated with the drug.

The dry blend of metformin could be prepared with hydrophilic polymer(s): hydroxypropyl methylcellulose and sodium carboxymethylcellulose, and optionally other excipients. The powder blend may be sifted through a screen of suitable fineness to remove or break up lumps. This screening also affords additional mixing. For large quantities of powder, twin shell blenders, double cone blenders, planetary mixers or the like may be used.

The blend could be wet granulated with water or with an aqueous dispersion of the binder. For granulation, water or an aqueous dispersion of the binder can be added to the blend while mixing. The powder mass is typically wetted with water or the binding solution until the mass has a suitable consistency. The wet mass is forced through 8 or 10-mesh screen, however for large quantities comminuting mills suitable for wet screening may be used.

Wet granules can be dried in trays or in fluidized bed dryer. In a drying step, a residual amount of moisture may be maintained in the granulation, to maintain the various granulation ingredients, such as the polymers, in a hydrated state. Also, residual moisture content can contribute to the reduction of static electric charge on the particles. The stability of the product containing moisture sensitive active ingredients may be related to the moisture content of the product. Residual moisture content of the granules can be less than about 6.0%. Residual moisture content of the granules can be between about 3.5 and about 6.0% by weight in some embodiments.

After drying, the granules are reduced in particle size, for example, by passing through a small mesh screen. After sizing, the granules can be lubricated and compressed to form tablets.

The invention will be further described in the following examples, which demonstrate general synthetic procedures, as well as specific preparations of some preferred formulations. The examples do not limit the scope of the invention described in the claims.

### EXAMPLES

In the examples, metformin tablets were prepared by processes described herein using hydrophilic polymers in a concentration of at least about 16% by weight of the composition which consists of 10-20% of hydroxypropyl methylcellulose with an average molecular weight in the range of 180,000 to 250,000, with a methoxy degree of substitution ranging from about 19 to about 24% and hydroxypropyl molar substitution ranging from about 7 to about 12% and from about 3 to about 10% sodium carboxymethylcellulose with a viscosity in the range of from about 400 to about 800 cps. Compositions of the tablets of Examples 1-6 are tabulated in Table 1. The sodium carboxymethylcellulose used in this particular example had viscosity of from about 400 to about 800 cps. The hydroxypropyl methylcellulose used in this example had an average molecular weight in the range of from about 180,000 to about 250,000, with a methoxy degree of substitution ranging from about 19 to about 24% and hydroxypropyl molar substitution ranging from about 7 to about 12%.

TABLE 1
Composition of Tablets of Examples 1-6

| | % w/w per Tablet | | | | | |
|---|---|---|---|---|---|---|
| Ingredient | Ex. 1 | Ex. 2 | Ex. 3 | Ex. 4 | Ex. 5 | Ex. 6 |
| Metformin hydrochloride | 68.0 | 73.0 | 69.0 | 72.7 | 68.0 | 49.5 |
| Sodium | 4.0 | 3.6 | 6.9 | 7.3 | 8.0 | 9.9 |
| carboxymethylcellulose | | | | | | |
| Hydroxypropyl | 12.0 | 12.7 | 12.4 | 12.7 | 12.0 | 18.8 |
| methylcellulose** | | | | | | |
| Binder | 1.6 | 1.8 | 1.7 | 1.8 | 1.6 | 1.5 |
| Diluent | 13.2 | 7.3 | 8.6 | 4.1 | 9.2 | 19.3 |
| Lubricant | 0.6 | 0.7 | 0.7 | 0.7 | 0.6 | 0.5 |
| Glidant | 0.6 | 0.7 | 0.7 | 0.7 | 0.6 | 0.5 |

The tablets were prepared by the following process:

1. Mix metformin with sodium carboxymethylcellulose, hydroxypropyl methylcellulose and diluent.

2. Granulate with a sufficient quantity of a solution of binder in water.

3. Pass the wet mass through a #10 BSS and dry in a fluidized bed dryer at about 60° C.

4. Pass the dry-mass through a #22 BSS, lubricate and compress into capsule shaped tablets.

Table 2 provides the in-vitro release profile of the controlled release tablets of metformin prepared by the composition and process of examples (1-6) in phosphate buffer pH 6.8 (900 ml), USP 2 at 50 rpm. The dissolution methodology for tablets consist of USP 2 with 10 mesh baskets being used as sinkers. Tablets are kept in the sinkers to prevent floating or sticking to the bottom. Paddle height is adjusted to 4.5 cm from the bottom of the vessel to prevent any hinderance to paddle rotation. 900 ml of the media is used (phosphate buffer pH 6.8) with a paddle rotation being maintained at 50 rpm.

The percent drug released was measured by techniques known to those of ordinary skill in the art for quantitative determination of drug present in solution, for example, by HPLC or reverse HPLC.

TABLE 2
Release profile of the controlled
release tablets of metformin prepared as per
Examples 1-6 in Phosphate buffer pH 6.8 (900 ml), USP 2 at 50 rpm.

| | % drug released | | | | | |
|---|---|---|---|---|---|---|
| Time (hr) | Ex. 1 | Ex. 2 | Ex. 3 | Ex. 4 | Ex. 5 | Ex. 6 |
| 0.5 | 22 | 20 | — | 19 | 22 | 23 |
| 1.0 | 32 | 27 | 38 | 27 | 32 | 31 |
| 2.0 | 48 | 38 | 54 | 38 | 46 | 45 |
| 4.0 | 64 | 58 | 74 | 59 | 65 | 60 |
| 6.0 | 74 | 69 | 88 | 69 | 78 | 72 |
| 8.0 | 83 | 81 | 98 | 80 | 84 | 83 |
| 10.0 | 90 | 88 | 101 | 87 | 91 | 90 |
| 12.0 | 92 | 95 | 104 | 96 | 96 | 92 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. Controlled-release metformin tablets comprising:
metformin; and
hydrophilic polymers comprising anionic and nonionic polymers in a ratio of about 1:1 to about 1:5, wherein at least about 16% by weight of the composition is the hydrophilic polymer.

2. The tablets of claim 1, wherein anionic polymer is sodium carboxymethylcellulose.

3. The tablets of claim 2, wherein sodium carboxymethylcellulose has a viscosity of from about 400 to about 800 cps.

4. The tablets of claim 2, wherein the concentration of sodium carboxymethylcellulose is from about 3 to about 10% by weight of the composition.

5. The tablets of claim 1 wherein nonionic polymer is hydroxypropyl methylcellulose.

6. The tablets of claim 5 wherein nonionic polymer is hydroxypropyl methylcellulose with an average molecular weight in the range of from about 180,000 to about 250,000, with a degree of methoxy substitution ranging from about 19 to about 24% and hydroxypropyl molar substitution ranging from about 7 to about 12%.

7. The tablets of claim 6 wherein hydroxypropyl methylcellulose has an average molecular weight of about 215,000.

8. The tablets of claim 5 wherein the concentration of hydroxypropyl methylcellulose is from about 10 to about 20% by weight of the composition.

9. The tablets of claim 1 further comprising excipients selected from diluents, binders, lubricants and glidants.

10. A process for the preparation of controlled release tablets of metformin, comprising the steps;
a) dry blending metformin with hydrophilic polymers comprising anionic and nonionic polymers in a ratio of about 1:1 to about 1:5, wherein at least about 16% by weight of the composition is the hydrophilic polymer;
b) granulating the blend;
c) drying and sizing the granules; and
d) compressing the granules to make tablets.

11. The process according to claim 10 wherein anionic polymer is sodium carboxymethylcellulose.

12. The process according to claim 11 wherein sodium carboxymethylcellulose has a viscosity of from about 400 to about 800 cps.

13. The process according to claim 12 wherein the concentration of sodium carboxymethylcellulose is from about 3 to about 10% by weight of the composition.

14. The process according to claim 10 wherein nonionic polymer is hydroxypropyl methylcellulose.

15. The process of claim 14 wherein nonionic polymer is hydroxypropyl methylcellulose with an average molecular weight in the range of from about 180,000 to about 250,000, with a degree of methoxy substitution ranging from about 19 to about 24% and hydroxypropyl molar substitution ranging from about 7 to about 12%.

16. The process of claim 15 wherein hydroxypropyl methylcellulose has an average molecular weight of about 215,000.

17. The process according to claim 14 wherein the concentration of hydroxypropyl methylcellulose is from about 10 to about 20% by weight of the composition.

18. The process according to claim 10 further comprising excipients selected from diluents, binders, lubricants and glidants.

19. The process according to claim 10 wherein the granulation is done with aqueous or non-aqueous solvents or dispersion of the binder.

20. The process according to claim 19 wherein the non-aqueous solvent is isopropyl alcohol.

21. The process according to claim 10 wherein the step c) granules are dried to a residual moisture content of between about 3.5 to about 6.0% by weight.

22. The process according to claim 10 wherein the tablet produced has an extended release up to about 12 hours.
